(19) United States
(12) Patent Application Publication
(10) Pub. No.: US 2025/0339088 A1
(43) Pub. Date: Nov. 6, 2025

(54) LASER SPECKLE IMAGING WITH NEAR INFRARED AUTOFLUORESCENCE TO MINIMIZE FALSE POSITIVE WITH LESS PERFUSED TISSUE

(71) Applicant: AI Biomed Corp., Santa Barbara, CA (US)

(72) Inventors: Adnan Subhi Abbas, Santa Barbara, CA (US); Nicholas F. Pergola, Lafayette, CO (US)

(21) Appl. No.: 19/193,005

(22) Filed: Apr. 29, 2025

Related U.S. Application Data
(60) Provisional application No. 63/640,892, filed on May 1, 2024.

(51) Int. Cl. A61B 5/00

(52) U.S. Cl. CPC A61B 5/4227; A61B 5/0071

(57) ABSTRACT

A tissue detection system includes a light source configured to illuminate tissue of interest. An imaging head including a detector is configured to acquire auto-fluorescence of the illuminated target tissue of interest responsive to the application of the light and generate one or more auto-fluorescence images of the target tissue of interest. A controller is configured to regulate operational control of the imaging head when acquiring, receiving, and processing images. If an intensity signal of the detected one or more auto-fluorescence images of the target tissue of interest leads to a determination of parathyroid tissue, the detector is configured to acquire laser speckle contrast images to determine the amount of perfusion of the target tissue of interest to distinguish well perfused parathyroid tissue having a low speckle contrast image from less perfused tissue having a high speckle contrast image. The less perfused tissue is identified a potential false positive.

### BACKGROUND

This application claims priority from U.S. Provisional Application Ser. No. 63/640,892, filed May 1, 2024, entitled “LASER SPECKLE IMAGING WITH NEAR INFRARED AUTOFLUORESCENCE TO MINIMIZE FALSE POSITIVE WITH LESS PERFUSED TISSUE”, the entire contents of which being incorporated by reference herein.

### TECHNICAL FIELD

The present disclosure relates to tissue detection and, more particularly, to systems and methods facilitating more accurate detection of tissue of interest at a surgical site.

### BACKGROUND OF RELATED ART

Many surgical procedures are performed at surgical sites on or within the body where the detection of tissue of interest via direct visualization techniques alone (e.g., using the human eye, a lens-based endoscope, a surgical video camera, etc.) is difficult due to obstructions, darkness, minimal or no contrast between different tissues, minimal or no visible distinction between different tissues, etc. Such surgical procedures may thus benefit from the use of enhanced visualization techniques such as, for example, fluorescence.

Since some materials, including certain tissues, fluoresce when stimulated with electromagnetic radiation (e.g., light at non-visible wavelengths), fluorescence can be used to highlight tissue of interest, thus facilitating detection of tissue of interest that may otherwise be difficult or impossible to detect solely by direct visualization techniques. The particular wavelength or wavelengths of electromagnetic radiation emitted and detected may depend upon the tissue or tissues of interest to be highlighted.

Coupling various fluorescence techniques (e.g., near infrared autofluorescence) with Laser Speckle Imaging (LSI) allows the surgeon to differentiate the blood flow and changes in blood flow through different tissue allowing the surgeon to distinguish tissue types. Since certain tissue types such as fat, e.g., brown fat, are less perfused that other well perfused tissue types, e.g., Parathyroid Gland (PTG), utilizing LSI in combination with NIRAF minimizes false positive readings when the system encounters less perfused tissue. Brown fat is one type of tissue that has fluorescence behavior similar to the PTG of interest, and thus the difference in perfusion of e.g. brown fat can assist in distinguishing it from PTG, which may not be possible via autofluorescence measurements alone.

### SUMMARY

To the extent consistent, any or all of the aspects detailed herein may be used in conjunction with any or all of the other aspects detailed herein.

Provided in accordance with aspects of the present disclosure is a tissue detection (or identification) system including a light source configured to emit a beam of light having a wavelength to illuminate a target tissue of interest. An imaging head having a detector is configured to acquire auto-fluorescence of the illuminated target tissue of interest responsive to the application of the light and generate one or more auto-fluorescence images of the target tissue of interest. A controller is configured to regulate operational control of the imaging head when acquiring, receiving, and processing images. If an intensity signal of the detected one or more auto-fluorescence images of the target tissue of interest leads to a determination of parathyroid tissue, the detector is configured to further (or simultaneously with the acquiring of the auto-fluorescence image) acquire laser speckle contrast images to determine the amount of perfusion of the target tissue of interest. This distinguishes well perfused parathyroid tissue having a low speckle contrast image from less perfused tissue having a high speckle contrast image. The less perfused tissue is identified a potential false positive as this could also indicate a non-perfused parathyroid tissue.

In aspects according to the present disclosure, the detector is a near infrared auto-fluorescence system. In other aspects according to the present disclosure, the detector is a near infrared auto-fluorescence system and a laser speckle contrast image system.

In aspects according to the present disclosure, the detector includes a moveable switching plate configuring to accommodate one or more filters and one or more irises, the moveable switching plate moveable between a first position wherein the one or more filters is positioned within an optical path of the detector allowing the detector to acquire auto-fluorescenced images, and a second position wherein the one or more irises is positioned within the optical path of the detector allowing the detector to acquire laser speckle contrast images.

In aspects according to the present disclosure, a linear actuator is disposed on the imaging head and is configured to coordinate movement of the moveable switching plate with the controller.

In aspects according to the present disclosure, the controller controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

Provided in accordance with aspects of the present disclosure is a tissue detection (or identification) system including a near infrared light source configured to illuminate a target tissue of interest. An imaging head including a detector is configured to acquire auto-fluorescence and laser speckle contrast images of the illuminated target tissue of interest responsive to the application of light from the light source. A controller is configured to regulate operational control of the imaging head when acquiring, receiving, and processing the images. If an intensity signal of the detected auto-fluorescence image of the target tissue of interest leads to a determination of parathyroid tissue, the controller is configured to assess the laser speckle contrast images to determine the amount of perfusion of the target tissue of interest to distinguish well perfused parathyroid tissue having a low speckle contrast image from less perfused tissue having a high speckle contrast image, the less perfused tissue being identified a potential false positive.

In aspects according to the present disclosure, the detector includes a moveable switching plate configuring to accommodate one or more filters and one or more irises. The moveable switching plate is moveable between a first position wherein the one or more filters is positioned within an optical path of the detector allowing the detector to acquire auto-fluoresced images, and a second position wherein the one or more irises is positioned within the optical path of the detector allowing the detector to acquire laser speckle contrast images

In aspects according to the present disclosure, a linear actuator is disposed on the imaging head and is configured to coordinate movement of the moveable switching plate with the controller.

In aspects according to the present disclosure, the controller controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

Provided in accordance with aspects of the present disclosure is a method for intraoperative assessment of parathyroid gland viability which includes illuminating a target tissue of interest with an infrared light source and acquiring auto-fluorescence images from a near infrared auto-fluorescence system. The method further includes determining if the target tissue of interest is auto-fluorescing wherein: if the target tissue of interest is not auto-fluorescing, illuminating a different target tissue of interest to acquire auto-fluorescence images of the different target tissue and determining if the different target tissue is auto-fluorescing and repeating the illuminating and acquiring steps until target tissue is auto-fluorescing; if the target tissue of interest is auto-fluorescing, acquiring images of the target tissue of interest from a laser speckle contrast imaging system to determine the amount of perfusion of the target tissue of interest wherein well perfused parathyroid tissue has low speckle contrast images while less perfused tissue has high speckle contrast images; and if the target tissue of interest auto-fluoresced was identified as less perfused tissue, identifying the target tissue of interest as a potential false positive.

In aspects according to the present disclosure, an imaging head houses a detector which includes the near infrared auto-fluorescence system and the laser speckle contrast imaging system. In other aspects according to the present disclosure, the method further includes moving a switching plate between a first position wherein one or more filters is positioned within an optical path of the detector allowing the detector to acquire auto-fluoresced images and a second position wherein one or more irises is positioned within the optical path of the detector allowing the detector to acquire laser speckle contrast images.

In aspects according to the present disclosure, the method further includes: controlling movement of the switching plate with a linear actuator disposed on the imaging head; and coordinating movement of the switching plate with a controller. In aspects according to the present disclosure, the controller also controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a tissue detection system in accordance with the present disclosure illustrated in use in relation to a patient and an operator, where an internal area of the patient is enlarged for reference;

FIG. 2 is a schematic illustration of a probe, emitter, detector, controller, and user interface of the tissue detection system of FIG. 1;

FIG. 3 is a block diagram of the probe of FIG. 2, the controller, the user interface, a camera system, a display, and a computing system of the tissue detection system of FIG. 1 illustrated in use in relation to a patient;

FIG. 4 is a block diagram of implementation of a machine learning algorithm by the computing system of the tissue detection system of FIG. 1;

FIG. 5 is a flow diagram of a method of detecting tissue in accordance with the present disclosure;

FIGS. 6A-6F schematically show a combined auto-fluorescence and laser speckle contrast imaging (LSCI) system for intraoperative assessment of parathyroid gland vascularity according to an embodiment of the present disclosure; and

FIG. 6G is schematic layout of the system shown in FIGS. 6A-6F.

### DETAILED DESCRIPTION

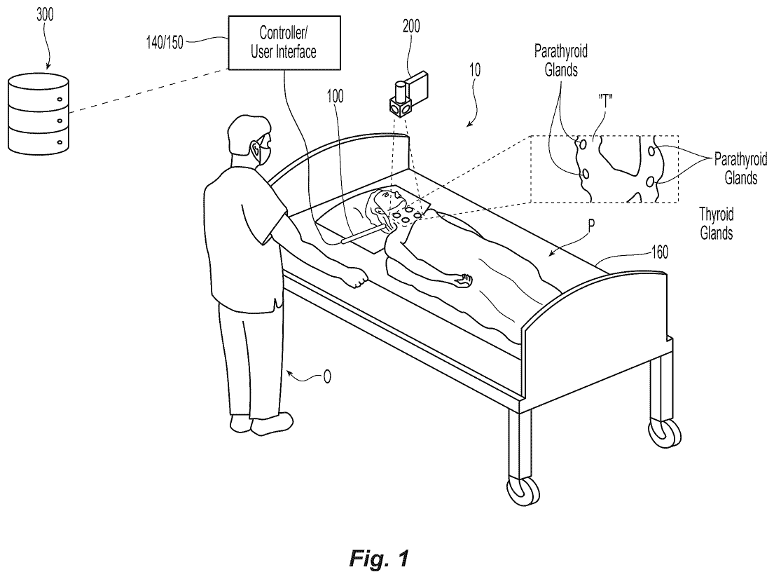

In the following description, well-known functions or constructions are not described in detail to avoid obscuring the present disclosure in unnecessary detail. Those skilled in the art will understand that the systems and methods of the present disclosure may be performed by one or more operators “O” (FIG. 1), which may be one or more human clinicians and/or one or more surgical robots.

The tissue detection or tissue identification systems and methods of the present disclosure may be utilized in surgical procedures to detect tissue (via affirmative or negative identification relative to surrounding tissue) and, if applicable, facilitate performing a surgical procedure on and/or around the detected tissue. For example, the tissue detection systems and methods of the present disclosure may be utilized to detect parathyroid tissue (e.g., within thyroid tissue), thyroid tissue, and/or other tissues in the neck region to facilitate removal or treatment of such tissue or surrounding tissue during surgery, or to avoid such tissue when removing or treating other tissue during surgery. However, although the aspects and features of the present disclosure are described hereinbelow with respect to detecting tissue in the neck region, e.g., parathyroid tissue and/or thyroid tissue, the aspects and features of the present disclosure are equally adaptable for use in the detection of different tissue and/or tissue at different anatomical locations. That is, although different instrumentation may be required to access different tissue and/or different anatomical locations, and although different settings, e.g., different electromagnetic radiation wavelengths, may be required to identify different tissue, the aspects and features of the present disclosure remain generally consistent regardless of the particular instrumentation and/or settings utilized.

Further, once a tissue is identified, in order to limit or reduce what is commonly referred to as a “false positive” by the various instrumentation of the tissue detections systems being utilized, additional instrumentation and methods may be required to minimize false detection of healthy parathyroid tissue.

Referring to FIG. 1, a tissue detection or identification system 10 provided in accordance with aspects of the present disclosure generally includes an optional probe 100, a controller 140, and a user interface 150. Tissue detection system 10 further includes a camera system 200 that can include a near-infrared (NIFR) camera to capture fluorescence (as still images and/or a video feed) from fluorescing tissue of interest and/or a visible camera (to capture still visible images and/or a visible video feed of tissue of interest). Further, tissue detection system 10 includes or is connected to a computing system 300 that communicates with controller 140, e.g., through a wired or wireless connection. Computing system 300 can include one or more computing devices (e.g., desktop computers, laptop computers, tablets, smartphones, servers (such as local servers, remote servers, and/or cloud servers), combinations thereof, and/or any other suitable computing devices) connected to one another and controller over a communication link such as a network accessible via the internet or an intranet.

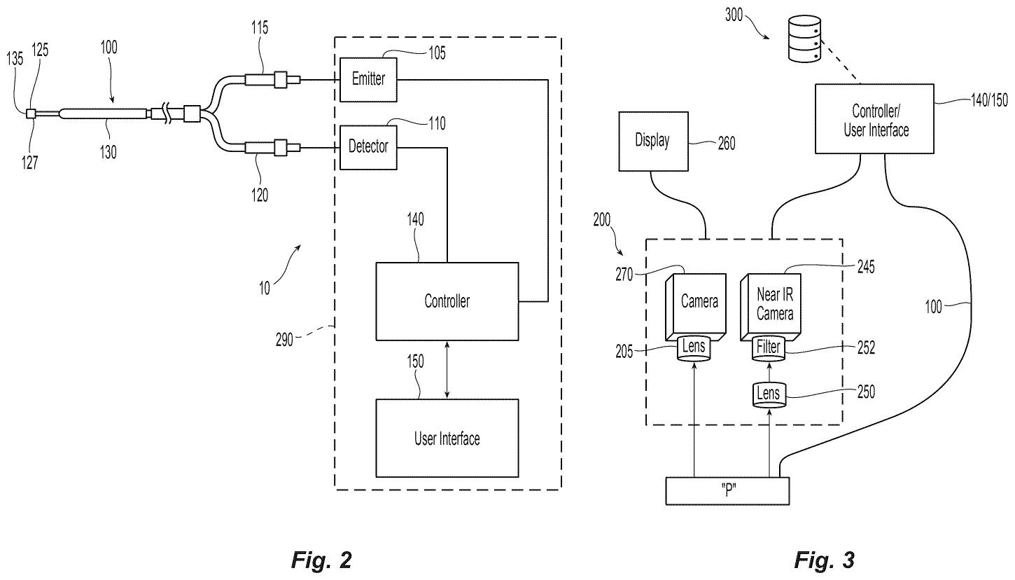

Referring still to FIG. 1, probe 100 can be positioned by an operator “O” relative to a patient “P” received on a surgical table 160. Although the operator “O” is shown as a human clinician, it is also contemplated that the operator “O” is a surgical robot. Probe 100 is configured to be maneuvered into position, e.g., by operator “O,” into contact or close proximity (e.g., within about 5 cm) and directed at tissue of interest such as parathyroid tissue “T” of patient “P.” Probe 100 operably connects to one or more emitters 105 (FIG. 2) configured to direct electromagnetic radiation from probe 100 to the tissue of interest to stimulate the tissue of interest such that any fluorescence produced by the stimulated tissue can be detected by one or more detectors 110 (FIG. 2), which may be operably coupled to probe 100, incorporated into or operably coupled to camera system 200, and/or separately provided.

Camera system 200, and as mentioned above, is configured to detect fluorescence and/or to obtain visible images. To this end, camera system 200 may include, for example, an IR camera 245 (FIG. 3), such as, for example, a NIFR camera, and/or a visible camera 270 (FIG. 3). Camera system 200 may be positioned spaced-apart from the surgical site as compared to probe 100, such that cameral system 200 may provide fluorescence detection and/or visible imaging over a relatively large field of view. In such aspects, combining use of camera system 200 with probe 100 enables fluorescence detection by camera system 200 to identify potentially fluorescing tissue over the relatively large field of view, and enables probe 100 to be used for fluorescence detection locally, within the relatively focused field of view thereof, at the location of each of the potentially fluorescing tissues, e.g., by positioning probe 100 in contact with or in close proximity (e.g., within about 5 cm) to the surface of each of the potentially fluorescing tissues, to enable confirmation as to whether the potentially fluorescing tissue identified by camera system 200 is indeed fluorescing. Tissue identification may also take place using a Parathyroid Detection System, e.g., the PTeye™ Probe sold by Medtronic. Laser speckle calculations may then be shown to indicate tissue perfusion. An NIFR camera may be used to detect the tip of the probe 100 (as being the area of interest to perform LSI.

Continuing with reference to FIG. 1, controller 140 and user interface 150 may be incorporated into a single integrated unit, may be physically connected or connectable with one another, or may be separate from one another. Controller 140 and/or user interface 150 can include a display or be connectable to a display, e.g., display 260 (FIG. 3), for displaying information obtained through use of tissue detection system 10 such as, for example, IR images of fluorescing tissue and/or visible images of tissue.

Controller 140 includes a processor to process data, a memory in communication with the processor to store data, and an input/output unit (I/O) to interface with other modules, units, and/or devices. The processor can include a central processing unit (CPU), a microcontroller unit (MCU), or any other suitable processor or processors. The memory can include and store processor-executable code, which when executed by the processor, causes controller 140 to perform various operations, e.g., such as receiving information, commands, and/or data, processing information and data, and transmitting or providing information/data to another device. To support various functions of controller 140, the memory can store information and data, such as instructions, software, values, images, and other data processed or referenced by the processor. Various types of RAM devices are envisioned. The I/O of controller 140 enables controller 140 to interface with other devices or components of devices utilizing various types of wired or wireless interfaces (e.g., a wireless transmitter/receiver (Tx/Rx)) compatible with typical data communication standards to enable communication between controller 140 and other devices, e.g., user interface 150, display 260 (FIG. 3), computing system 300, etc.

Any known user interface 150 is envisioned that enables the input of information, e.g., to control the operation of system 10, and/or to output information, e.g., regarding the status and/or result of the operation of system 10. With additional reference to FIG. 2, probe 100, in aspects, may include one or more probe bodies 130 each including one or more emitter optical fibers 115 coupled to one or more emitters 105 and/or one or more detector optical fibers 120 coupled to one or more detectors 110. Additional or alternative detection may be provided by camera system 200 (FIG. 1), as detailed below. Emitter 105 and detector 110 may be integrated into a single unit, e.g., a console 290 including a housing, or may be separate from one another. For example, console 290 may include emitter 105, detector 110, controller 140 and/or user interface 150 incorporated therein or thereon.

Emitter 105 is configured to emit electromagnetic radiation at a particular wavelength or within a particular wavelength range, e.g., via tuning and/or equipment selection, through emitter optical fiber 115 and out a distal end portion 135 of probe body 130 (either axially therefrom, transversely therefrom, or in any other suitable direction or directions including adjustable directions) in order to stimulate fluorescence of a particular tissue or tissues of interest. With respect to identification of parathyroid tissue, for example, emitter 105 (with or without the use of one or more optical elements 125 disposed at the output end of emitter optical fiber 115 at distal end portion 135 of probe body 130) may be configured to emit electromagnetic radiation in the form of laser energy at a wavelength of about 785 nm to facilitate auto-fluorescence of parathyroid tissue. Emitter 105, at least for use in identifying parathyroid tissue, may be a narrow band source such as a laser (e.g., a solid-state laser, a laser diode, etc.) or other suitable source whose electromagnetic radiation output wavelength is at or near a narrow band around about 785 nm.

Controller 140 can be used to control transmission, e.g., activate/deactivate, control the wavelength, intensity, etc., of the electromagnetic radiation from emitter 105 to tissue of interest (via emitter optical fiber 115). User interface 150 can be used to interact with and control operation of the controller 140 (e.g., to set parameters and/or activate/deactivate), which in turn controls emitter 105.

Detector 110 is configured to detect fluorescence of the tissue of interest (as a result of the electromagnetic radiation emitted to stimulate the tissue of interest) collected at distal end portion 135 of probe body 130 and transmitted through detector optical fiber 120 to detector 110. Detector 110 is further configured to process the received fluorescence signal. Controller 140 may be utilized to control and/or facilitate processing of the detected fluorescence signal at detector 110. With respect to detection of parathyroid tissue, for example, detector 110 may be configured to process the fluorescence signal, which for parathyroid tissue undergoing auto-fluorescence is at wavelengths ranging from about 808 nm to about 1000 nm. Detector 110 may be an avalanche photodiode or other near IR detector, a 2D array of IR detectors, or other suitable detector, and may be used in concert with one or more optical elements 127, e.g., a longpass (highpass) optical filter, such that radiation wavelengths above the source wavelength (for instance, above about 800 nm, e.g., ranging from about 808 to about 1000 nm) can be detected with minimal interference from other non-relevant wavelengths of electromagnetic radiation, e.g., such as from ambient light.

A detected fluorescence signal, e.g., obtained and processed by detector 110, for a tissue of interest may be evaluated by controller 140 by comparing the detected fluorescence signal with a baseline fluorescence signal to determine if the detected fluorescence signal is indicative of the presence of a particular tissue, or may be processed in any other suitable manner. Details with respect to systems and methods using autofluorescence for discriminating parathyroid tissue from thyroid tissue or other tissues in a neck region are described in U.S. Pat. No. 9,687,190 titled “Intra-Operative Use of Fluorescence Spectroscopy and Applications of Same,” the entire contents of which are hereby incorporated by reference herein. As disclosed therein, when the thyroid tissue and the parathyroid tissue are exposed to radiation in a narrow wavelength range of about 785 nm, which is just outside the visible light range, both the thyroid tissue and the parathyroid tissue produce auto-fluorescence in a wavelength range above about 800 nm, sometimes centered at about 822 nm (the wavelength range above about 800 nm is also not visible).

However, the intensity of the auto-fluorescence of the parathyroid tissue is significantly higher than that of the thyroid tissue, enabling distinction between these two tissues and, thus, detection of the parathyroid tissue within the thyroid tissue. More specifically, the detection of the parathyroid tissue within the thyroid tissue may be determined by controller 140, for example, based on a ratio of the intensity of the detected fluorescence signal to the intensity of the baseline fluorescence signal. With respect to areas where the intensity exceeds a threshold or other criteria, those areas may be identified as parathyroid tissue. These systems and methods may also be applied for use in detecting other tissues (with appropriate adjustment of the wavelengths and baseline signals). Other suitable systems methods for detecting tissue, e.g., parathyroid tissue, are also contemplated.

In addition to detecting tissue (for example, parathyroid tissue within thyroid tissue) based on a comparison of the detected fluorescence signal with the baseline fluorescence signal (e.g., using a ratio of the intensities thereof), controller 140 may be configured to determine a confidence associated with the tissue detection. This confidence may be, in aspects, a numerical value indicating a confidence in the tissue detection such as, for example: a confidence number on a numerical scale (e.g., 1-10); a confidence percentage on a percentile scale (e.g., 0-100%); or any other suitable numerical value. Alternatively or additionally, the confidence may be a categorical determination indicating a confidence in the tissue detection such as, for example: a binary determination of sufficient or insufficient (YES/NO) confidence in the tissue detection; a determination of a confidence level, e.g., high confidence, moderate confidence, or low confidence in the tissue detection, or any other suitable categorical determination of confidence associated with the tissue detection. The confidence of tissue detection is described in detail in PCT Publication Serial No. WO2024250274A1 entitled: TISSUE DETECTION SYSTEMS AND METHODS the entire contents of which being incorporated by reference herein.

The confidence in the tissue detection, as detailed below, is utilized to determine whether further action is required to enable accurate detection of the tissue of interest. If confidence meets a threshold level tissue detection is determined to have been accomplished with sufficient confidence such that no further detection or confirmation is required. On the other hand, where the confidence is below the confidence threshold, tissue detection is determined to have been accomplished with insufficient confidence such that further detection or confirmation is required. Many tissue detection and confidence techniques are described in the above-mentioned PCT Publication Serial No. WO2024250274A1, e.g., via controller 140, in first manner, e.g., using fluorescence, the further detection or confirmation may be performed remotely, e.g., via computing device 300 (FIGS. 1 and 3), and/or in a second, different manner, e.g., using image processing, utilizing a traditional algorithm(s), utilizing a machine learning algorithm(s).

Utilization of the above tissue detection and confidence techniques together with the various components of the tissue detection system 10 are described in detail in the above-identified PCT Publication Serial No. WO2024250274A1. Any one or more of these techniques may be implemented with the tissue detection system of the present disclosure.

In accordance with another embodiment of the present disclosure, the tissue detection system 10 can also be configured to include a Laser Speckle Contrast Imaging (LSCI) system which works in tandem with the tissue detections systems mentioned above (in particular, near infrared auto-fluorescence systems) to target and identify target tissue of interest using one or more imaging heads to obtain fluorescence images of the tissue and a series of speckle contrast images of the tissue. Details relating to using these systems (e.g., NIRAF system) in combination with a LSCI system to identify target tissue of interest are discussed in U.S. patent application Ser. No. 17/289,323, the entire contents of which being incorporated by reference herein.

Briefly, LSCI refers to a technique for imaging vascular flow through tissue, e.g., parathyroid gland (PTG). LSCI utilizes intrinsic tissue contrast from dynamic light scattering and provides a relatively simple technique for visualizing detailed spatiotemporal dynamics of blood flow changes in real-time. Laser speckle is the random interference pattern produced when coherent light scatters from a random medium and can be imaged onto a detector. Motion from scattering particles, such as red blood cells in the vasculature, leads to spatial and temporal variations in the speckle pattern. Speckle contrast analysis quantifies the local spatial variance, or blurring, of the speckle pattern that results from blood flow. Areas with greater motion have more rapid intensity fluctuations and therefore have more blurring of the speckles during the camera exposure time. LSCI is used to quantify relative changes in blood flow.

The LSCI technique analyzes the interference pattern which fluctuates depending on how fast particles are moving. Blurring of the speckle pattern occurs when the motion is fast relative to the integration time of the detector. Analyzing this spatial blurring provides contrast between regions of faster motion versus slower motion and forms the basis of LSCI. This technique is sensitive to microvascular perfusion and has been employed in a variety of tissues where the vessels of interest are generally superficial.

Prior tissue detection systems include one or more components and techniques for increasing confidence in the system that the identity of the tissue of interest is accurate. In other words, once tissue is identified using one or more of the tissue detections systems described herein and referenced herein, various techniques can be employed to increase the confidence level of the tissue identity so that the surgeon may reliably proceed with the surgery. As mentioned above, other areas of concern include the so-called “false positive” identification of tissue which can also lead to surgical interruptions.

Recently, it has been found utilizing an LSCI system in one specific area of concern relating to distinguishing healthy parathyroid tissue from potentially false positive identified, less perfused tissue, e.g., brown adipose tissue or brown fat. More specifically, the LSCI system is particularly adapted to work in tandem with a NIRAF system such as the PTeye™ Parathyroid Detection System sold by Medtronic to reduce the occurrence false positives or to distinguish healthy parathyroid gland tissue (PGT) from less perfused tissue or glands, e.g., brown fat or brown adipose tissue.

It has been found that brown adipose tissue presents a similar NIRAF intensity and signal (or pattern) as healthy, well perfused PGT. The similarity of NIRAF intensity and signal (or pattern) between normal PGT and brown adipose tissue can be misleading during PGT localization in patients using simply NIRAF imaging. However, adding or combining LSCI imaging which shows intensity patterns of tissue perfusion (and/or rates of tissue perfusion with the images) from the NIRAF allows the surgeon (or system) to differentiate the brown adipose tissue from the PGT. False positive fluorescence from brown adipose tissue can be identified to distinguish potential false positive readings from viable PTG.

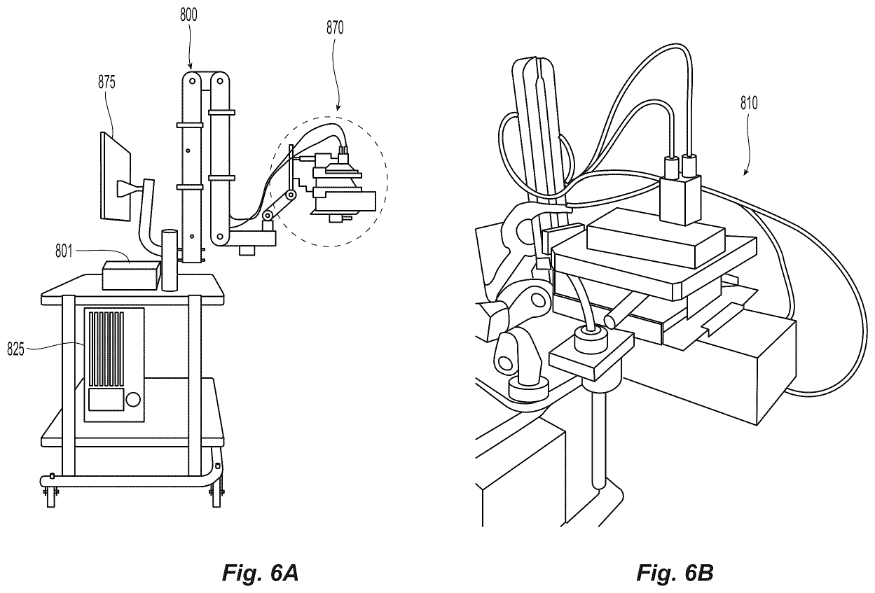

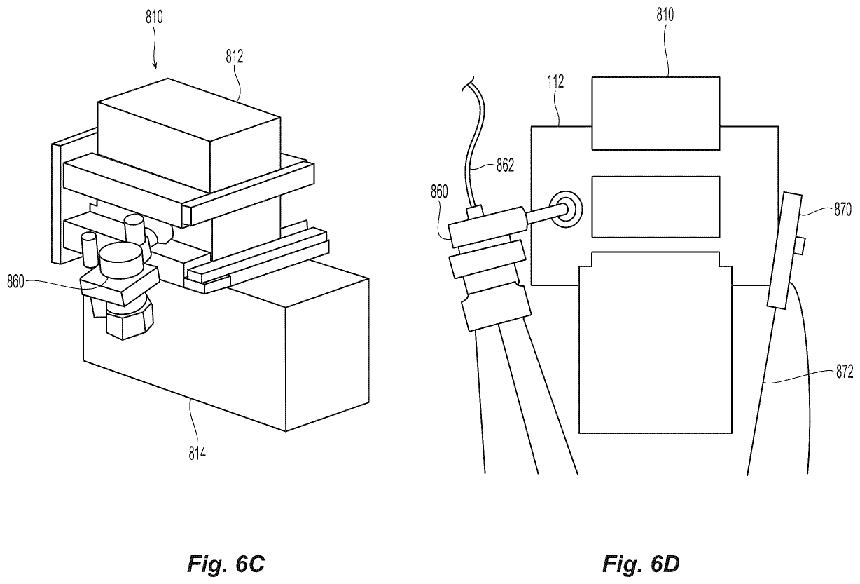

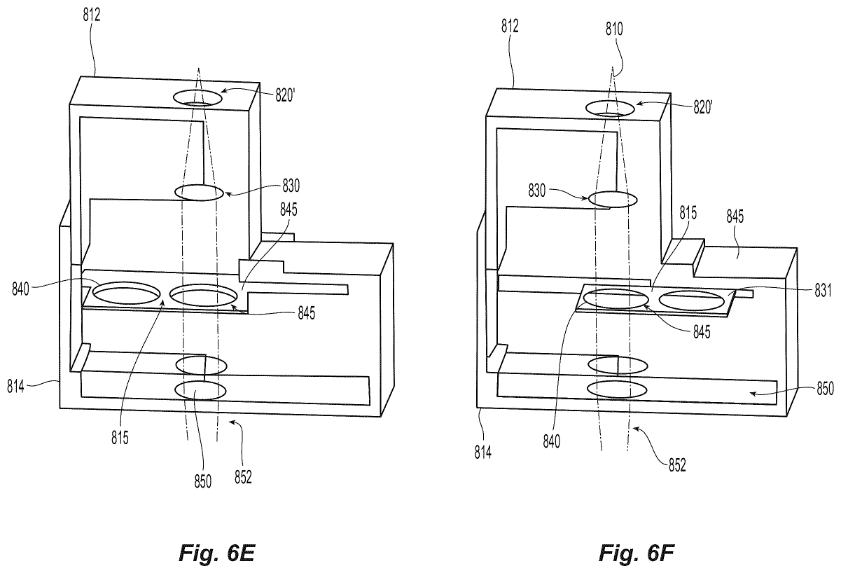

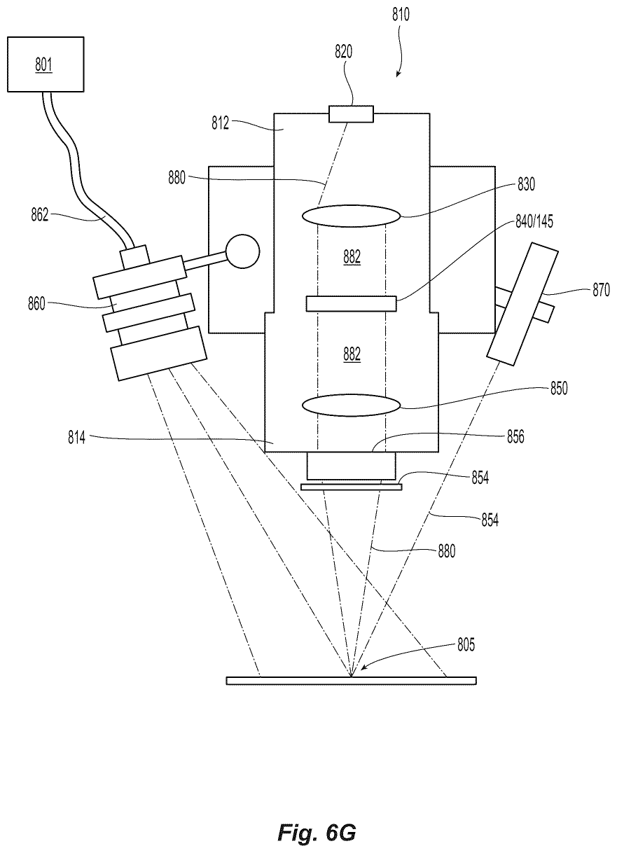

Referring to FIGS. 6A-6G, the combined NIRAF imaging and LSCI system 800 is shown according to one embodiment of the present disclosure. The system 800 includes a light source 801 (e.g., an infrared laser) for emitting a beam of light (at a wavelength of about 785 nm) to illuminate a target of interest 805; and an imaging head 810 positioned over the target of interest 805 for acquiring auto-fluorescence images and LSCI images of light from the illuminated target of interest 805 responsive to the illumination.

The imaging head 810 includes a detector 820 disposed in a top portion 812 of the image head 810 for individually acquiring the auto-fluorescence images and the LSCI images and a first lens 850 and a second lens 830 positioned in an optical path 882. The first lens 850 collects the light from the illuminated target of interest 805 in the surgical field and the second lens 830 focuses the collected light towards the detector 820. Various sets of lenses 830, 850 with different focal lengths (or ratios of focal lengths) are envisioned.

The imaging head 810 also has a movable switching plate 815 (FIGS. 6E and 6F) accommodating filters 840 and an iris 845, as shown in FIGS. 6E-6G, located between the first lens 850 and the second lens 830. The movable switching plate 815 is operably movable between a first position wherein the filters 840 are positioned in the optical path 882 (FIG. 6E) enabling the detector 820 to acquire auto-fluorescence images, and a second position wherein the iris 845 is positioned in the optical path (FIG. 6F) and the detector 820 acquires the LSCI images. It is envisioned that any long-pass or band-pass filters between the range of about 800 nm to about 830 nm may be utilized.

The imaging head 810 may include a linear actuator 831 which is configured to move the movable switching plate 815 between the first position and the second position. In addition, the imaging head 810 may include a focus tunable lens 852 disposed in a bottom portion 814 of the image head 810 and positioned between the target of interest 805 and the first lens 850 in the optical path 882 for focusing light 880 from the illuminated target of interest 805 in a surgical field. A first linear polarizer 854 may be positioned in the optical path between the focus tunable lens 852 and the target of interest 805.

The detector 820 includes one or more cameras, e.g., near infrared auto-fluorescence camera 820′ of FIGS. 6E and 6F, an infrared camera, a near-infrared camera, a charge-coupled device (CCD) camera and/or a metal oxide semiconductor (CMOS) camera.

The system 800 may further include one or more laser pointers 870 arranged in relation to the detector 820 such that a beam 872 of the laser pointer 870 is co-localized with a center of the field of view of the detector 820 at a distance, e.g., two laser pointers 870 attached on the sides of the imaging head 810 guide a surgeon in positioning the imaging head 810 so that the target of interest 805 in roughly in the center of the field of view and in focus when imaging, as shown in FIG. 6G. A lens tube 860 containing lenses may be arranged in relation to the target of interest 805. The light source 801 is optically coupled to the lens tube 860 via cable 862 for illuminating the target of interest 805.

A controller 825 (alternatively computer) is configured to control operations of the imaging head 810 for acquiring the auto-fluorescence and LSCI images of the illuminated target of interest 805, receiving the acquired auto-fluorescence and LSCI images from the detector 820, and processing the acquired auto-fluorescence and LSCI images to obtain speckle contrast images for the intraoperative assessment of parathyroid gland viability, e.g., a perfused parathyroid gland has low speckle contrast while a devascularized parathyroid gland or less perfused tissue, e.g., brown adipose fat has high speckle contrast. A display 875 displays the speckle contrast images of the target tissue of interest.

In one envisioned embodiment, two cameras (not shown) may be utilized with a beamsplitter (not shown) placed in the optical/detection path to simultaneously collect auto-fluorescence images and LSCI images.

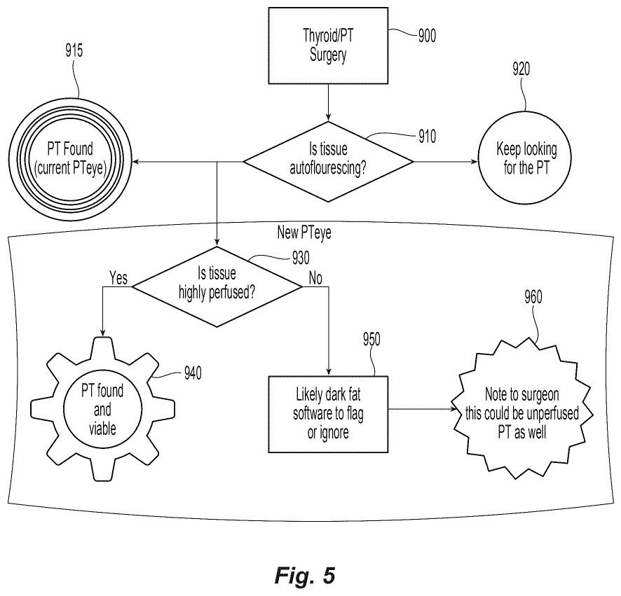

In another aspect of the disclosure, a method for intraoperative assessment of parathyroid gland viability for guidance in a surgery is shown in the flow diagram of FIG. 5 and includes: providing a beam of light to illuminate a target tissue of interest (900); acquiring auto-fluorescence images and determining if the target tissue of interest is auto-fluorescing (910); if the target tissue of interest is not auto-fluorescing, illuminating different target tissue of interest to acquire auto-fluorescence images of the different tissue and determining if the different tissue is auto-fluorescing (920); if the target tissue of interest is auto-fluorescing, current tissue detection systems, e.g., PTeye™, made a determination of viable parathyroid tissue (915) but there was a tendency of false positive identification with less perfused tissue, e.g., brown adipose tissue; with the presently disclosed method and system, step (930) acquires (LSCI) images of the target tissue of interest responsive to the illumination and determines the amount of perfusion of the target tissue of interest—wherein well perfused parathyroid gland has low speckle contrast (indicating viable parathyroid tissue—940) while less perfused tissue, e.g., brown adipose fat has high speckle contrast (indicating it is not parathyroid tissue 950); and if the target tissue of interest auto-fluoresced and was identified as less perfused tissue, the tissue is identified as a potential false positive (960) (with a note to the surgeon that the tissue of interest could be unperfused parathyroid tissue).

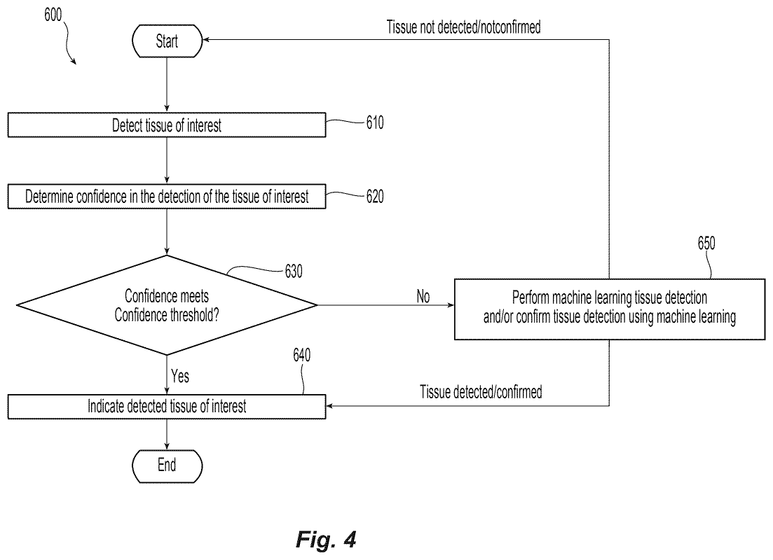

Turning back to FIG. 4, a method 600 of detecting tissue confidence is also disclosed and may be utilized as part of an additional step the method 900 to further surgical confidence. Initially, at 610, tissue of interest is detected, e.g., using fluorescence, auto-fluorescence, and LSCI as detailed above or in any other suitable manner. At 620, a confidence in the detection of tissue performed at 610 is determined. If the confidence meets or exceeds a confidence threshold, “YES” at 630, the method proceeds to 640 where the detected tissue of interest is indicated to the operator, e.g., via display, highlighting, audible tone, and/or other suitable indication.

If, on the other hand, the confidence does not meet the confidence threshold, “NO” at 630, the method proceeds to 650, wherein machine learning tissue detection is performed and/or the tissue detection is confirmed using machine learning. If the tissue is detected using machine learning tissue detection and/or if the tissue detection is confirmed using machine learning, “TISSUE DETECTED/CONFIRMED” at 650, the method proceeds to 640 where the detected tissue of interest is indicated to the operator, e.g., via display, highlighting, audible tone, and/or other suitable indication. If, on the other hand, the tissue is not detected using machine learning tissue detection and/or if the tissue detection is not confirmed using machine learning, “TISSUE NOT DETECTED/NOT CONFIRMED” at 650, the method reverts to the start, requiring re-starting of method 600 in order to detect the tissue of interest. An error may additionally or alternately be output in this situation.

A tissue detection system includes a light source configured to emit a beam of light having a wavelength to illuminate a target tissue of interest; an imaging head including a detector configured to acquire auto-fluorescence of the illuminated target tissue of interest responsive to the application of the light and generate one or more auto-fluorescence images of the target tissue of interest; and a controller configured to regulate operational control of the imaging head when acquiring, receiving, and processing images, wherein if an intensity signal of the detected one or more auto-fluorescence images of the target tissue of interest leads to a determination of parathyroid tissue, the detector is configured to at least one of further or simultaneously acquire laser speckle contrast images to determine the amount of perfusion of the target tissue of interest to distinguish well perfused parathyroid tissue having a low speckle contrast image from less perfused tissue having a high speckle contrast image, the less perfused tissue being identified a potential false positive.

In aspects in accordance with the present disclosure, the detector is a near infrared auto-fluorescence system.

In aspects in accordance with the present disclosure, the detector is a near infrared auto-fluorescence system and a laser speckle contrast image system.

In aspects in accordance with the present disclosure, the detector includes a moveable switching plate configuring to accommodate at least one filter and at least one iris, the moveable switching plate moveable between a first position wherein one of the at least one filters is positioned within an optical path of the detector allowing the detector to acquire auto-fluoresced images, and a second position wherein one of the at least one irises is positioned within the optical path of the detector allowing the detector to acquire laser speckle contrast images.

In aspects in accordance with the present disclosure, a linear actuator is disposed on the imaging head and configured to coordinate movement of the moveable switching plate with the controller.

In aspects in accordance with the present disclosure, the controller controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

A tissue detection system, includes a near infrared light source configured to illuminate a target tissue of interest; an imaging head including a detector configured to acquire auto-fluorescence and laser speckle contrast images of the illuminated target tissue of interest responsive to the application of light from the light source; and a controller configured to regulate operational control of the imaging head when acquiring, receiving, and processing the images, wherein if an intensity signal of the detected auto-fluorescence image of the target tissue of interest leads to a determination of parathyroid tissue, the controller is configured to assess the laser speckle contrast images to determine the amount of perfusion of the target tissue of interest to distinguish well perfused parathyroid tissue having a low speckle contrast image from less perfused tissue having a high speckle contrast image, the less perfused tissue being identified a potential false positive.

In aspects in accordance with the present disclosure, the detector includes a moveable switching plate configuring to accommodate at least one filter and at least one iris, the moveable switching plate moveable between a first position wherein one of the at least one filters is positioned within an optical path of the detector allowing the detector to acquire auto-fluoresced images, and a second position wherein one of the at least one irises is positioned within the optical path of the detector allowing the detector to acquire laser speckle contrast images.

In aspects in accordance with the present disclosure, a linear actuator is disposed on the imaging head and configured to coordinate movement of the moveable switching plate with the controller.

In aspects in accordance with the present disclosure, the controller controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

A method for intraoperative assessment of parathyroid gland viability, includes: illuminating a target tissue of interest with an infrared light source; and acquiring auto-fluorescence images from a near infrared auto-fluorescence system and determining if the target tissue of interest is auto-fluorescing, wherein: if the target tissue of interest is not auto-fluorescing, illuminating a different target tissue of interest to acquire auto-fluorescence images of the different target tissue and determining if the different target tissue is auto-fluorescing and repeating the illuminating and acquiring steps until target tissue is auto-fluorescing; if the target tissue of interest is auto-fluorescing, acquiring images of the target tissue of interest from a laser speckle contrast imaging system to determine the amount of perfusion of the target tissue of interest wherein well perfused parathyroid tissue has low speckle contrast images while less perfused tissue has high speckle contrast images; and if the target tissue of interest auto-fluoresced was identified as less perfused tissue, identifying the target tissue of interest as a potential false positive.

In aspects in accordance with the present disclosure, an imaging head houses a detector which includes the near infrared auto-fluorescence system and the laser speckle contrast imaging system.

In aspects in accordance with the present disclosure, the method includes moving a switching plate between a first position wherein at least one filter is positioned within an optical path of the detector allowing the detector to acquire auto-fluoresced images, and a second position wherein at least one iris is positioned within the optical path of the detector allowing the detector to acquire laser speckle contrast images.

In aspects in accordance with the present disclosure, the method includes controlling movement of the switching plate with a linear actuator disposed on the imaging head; and coordinating movement of the switching plate with a controller.

In aspects in accordance with the present disclosure, the linear actuator also controls operations of the imaging head for: acquiring the auto-fluorescence and laser speckle contrast images of the illuminated target tissue of interest; receiving the acquired auto-fluorescence and laser speckle contrast images from the detector; and processing the acquired auto-fluorescence and laser speckle contrast images to obtain speckle contrast images for the assessment of target tissue of interest identification.

While several aspects of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope of the claims appended hereto.

What is claimed is:

1. A tissue detection system, comprising:
a light source configured to emit a beam of light having a wavelength to illuminate a target tissue of interest;
an imaging head including a detector configured to acquire auto-fluorescence of the illuminated target tissue of interest responsive to the application of the light and generate one or more auto-fluorescence images of the target tissue of interest; and
a controller configured to regulate operational control of the imaging head when acquiring, receiving, and processing images,
wherein if an intensity signal of the detected one or more auto-fluorescence images of the target tissue of interest leads to a determination of parathyroid tissue, the detector is configured to at least one of further or simultaneously acquire laser speckle contrast images to determine the amount of perfusion of the target tissue of interest to distinguish well perfused parathyroid tissue having a low speckle contrast image from less perfused tissue having a high speckle contrast image, the less perfused tissue being identified a potential false positive.

2. The tissue detection system according to claim 1, wherein the detector is a near infrared auto-fluorescence system.

3. The tissue detection system according to claim 2, wherein the detector is a near infrared auto-fluorescence system and a laser speckle contrast image system.

4. The tissue detection system according to claim 3, wherein the detector includes a moveable switching plate configuring to accommodate at least one filter and at least one iris, the moveable switching plate moveable between a first position wherein one of the at least one filters is positioned within an optical path of the detector allowing the detector to acquire auto-fluoresced images, and a second position wherein one of the at least one irises is positioned within the optical path of the detector allowing the detector to acquire laser speckle contrast images.

5. The tissue detection system according to claim 4, further comprising a linear actuator disposed on the imaging head and configured to coordinate movement of the moveable switching plate with the controller.

6. The tissue detection system according to claim 1, wherein the controller controls operations of the imaging head for:
acquiring the auto-fluorescence and laser speckle contrast images of the illuminated target tissue of interest;
receiving the acquired auto-fluorescence and laser speckle contrast images from the detector; and
processing the acquired auto-fluorescence and laser speckle contrast images to obtain speckle contrast images for the assessment of target tissue of interest identification.

7. A tissue detection system, comprising:
a near infrared light source configured to illuminate a target tissue of interest;
an imaging head including a detector configured to acquire auto-fluorescence and laser speckle contrast images of the illuminated target tissue of interest responsive to the application of light from the light source; and
a controller configured to regulate operational control of the imaging head when acquiring, receiving, and processing the images,
wherein if an intensity signal of the detected auto-fluorescence image of the target tissue of interest leads to a determination of parathyroid tissue, the controller is configured to assess the laser speckle contrast images to determine the amount of perfusion of the target tissue of interest to distinguish well perfused parathyroid tissue having a low speckle contrast image from less perfused tissue having a high speckle contrast image, the less perfused tissue being identified a potential false positive.

8. The tissue detection system according to claim 7, wherein the detector includes a moveable switching plate configuring to accommodate at least one filter and at least one iris, the moveable switching plate moveable between a first position wherein one of the at least one filters is positioned within an optical path of the detector allowing the detector to acquire auto-fluoresced images, and a second position wherein one of the at least one irises is positioned within the optical path of the detector allowing the detector to acquire laser speckle contrast images.

9. The tissue detection system according to claim 8, further comprising a linear actuator disposed on the imaging head and configured to coordinate movement of the moveable switching plate with the controller.

10. The tissue detection system according to claim 7, wherein the controller controls operations of the imaging head for:
acquiring the auto-fluorescence and laser speckle contrast images of the illuminated target tissue of interest;
receiving the acquired auto-fluorescence and laser speckle contrast images from the detector; and
processing the acquired auto-fluorescence and laser speckle contrast images to obtain speckle contrast images for the assessment of target tissue of interest identification.

11. A method for intraoperative assessment of parathyroid gland viability, comprising:
illuminating a target tissue of interest with an infrared light source; and
acquiring auto-fluorescence images from a near infrared auto-fluorescence system and determining if the target tissue of interest is auto-fluorescing, wherein:
if the target tissue of interest is not auto-fluorescing, illuminating a different target tissue of interest to acquire auto-fluorescence images of the different target tissue and determining if the different target tissue is auto-fluorescing and repeating the illuminating and acquiring steps until target tissue is auto-fluorescing;
if the target tissue of interest is auto-fluorescing, acquiring images of the target tissue of interest from a laser speckle contrast imaging system to determine the amount of perfusion of the target tissue of interest wherein well perfused parathyroid tissue has low speckle contrast images while less perfused tissue has high speckle contrast images; and
if the target tissue of interest auto-fluoresced was identified as less perfused tissue, identifying the target tissue of interest as a potential false positive.

12. The method for intraoperative assessment of parathyroid gland viability according to claim 11, wherein an imaging head houses a detector which includes the near infrared auto-fluorescence system and the laser speckle contrast imaging system.

13. The method for intraoperative assessment of parathyroid gland viability according to claim 12, further comprising:
moving a switching plate between a first position wherein at least one filter is positioned within an optical path of the detector allowing the detector to acquire auto-fluoresced images, and a second position wherein at least one iris is positioned within the optical path of the detector allowing the detector to acquire laser speckle contrast images.

14. The method for intraoperative assessment of parathyroid gland viability according to claim 13, further comprising:
controlling movement of the switching plate with a linear actuator disposed on the imaging head; and
coordinating movement of the switching plate with a controller.

15. The method for intraoperative assessment of parathyroid gland viability according to claim 14, wherein the linear actuator also controls operations of the imaging head for:
acquiring the auto-fluorescence and laser speckle contrast images of the illuminated target tissue of interest;
receiving the acquired auto-fluorescence and laser speckle contrast images from the detector; and
processing the acquired auto-fluorescence and laser speckle contrast images to obtain speckle contrast images for the assessment of target tissue of interest identification.
